SEQUENCE LISTING (2 sequences)

SEQ ID NO: 1 - DNA, 40 bp, synthetic construct
  source 1..40: mol_type = other DNA
ccaaggcctgcaagggaaccaaggacacagtttttttttt

SEQ ID NO: 2 - DNA, 54 bp, synthetic construct
  source 1..54: mol_type = other DNA
acgggccacatcaactcattgatagacaatgcgtccactgcccgtttttttttt

(19) United States
(12) Patent Application Publication
(10) Pub. No.: US 2026/0191986 A1
(43) Pub. Date: Jul. 9, 2026

(54) LIPOSOME-BASED DRUG CARRIER FOR IMMUNE ANTICANCER THERAPY AND METHOD FOR PRODUCING SAME

(71) Applicant: KONKUK UNIVERSITY INDUSTRIAL COOPERATION CORP, Seoul (KR)

(72) Inventor: Dong-eun KIM, Seoul (KR)

(73) Assignee: KONKUK UNIVERSITY INDUSTRIAL COOPERATION CORP, Seoul (KR)

(21) Appl. No.: 19/134,113

(22) PCT Filed: Jun. 22, 2023
(86) PCT No.: PCT/KR2023/008696

§ 371 Date: May 29, 2025

(30) Foreign Application Priority Data
Dec. 14, 2022 (KR) 10-2022-0174798

(51) Int. Cl. A61K 47/69; A61K 31/138; A61K 31/337; A61K 31/555; A61K 31/704; A61K 33/243; A61K 47/54; A61P 35/00; C12N 15/113

(52) U.S. Cl. CPC A61K 47/6911; A61K 31/138; A61K 31/337; A61K 31/555; A61K 31/704; A61K 33/243; A61K 47/549; A61P 35/00; C12N 15/1136; C12N 15/1137; C12N 15/1138; C12N 2310/14; C12Y 113/11052; C12Y 114/13039; C12Y 305/03001

(57) ABSTRACT

The present disclosure relates to a liposome-based drug carrier for immune anticancer therapy, which includes a liposome; an aptamer which is a target-specific ligand for cancer cells conjugated on the surface of the liposome; and an immunogenic chemo-anticancer agent and siRNA, an immunosuppressive protein inhibitor, which are co-loaded to the liposome. Accordingly, it is possible to induce a continuous tumor-specific anticancer immune response in cancer patients having a low response rate to a standalone immunogenic anticancer agent therapy, and to reduce side effects due to non-specific drug delivery, thereby increasing the effects of immune anticancer therapy.

### TECHNICAL FIELD

The present disclosure relates to a drug carrier and a method for preparing the same, more specifically to a liposome-based drug carrier for immune anticancer therapy and a method for preparing the same.

### BACKGROUND ART

Immune anticancer therapy is a new cancer therapy wherein immune cells attack the cancer cells of a patient using the body's own immune system. However, there is a limitation in that the efficacy is only about 5-30% in total cancer patients, or tolerance may occur. One of the major factors that determine the efficacy of immune anticancer therapy is the tumor microenvironment (TME). It is known that therapeutic efficacy is very low under the environment where the amount of antigen-presenting cells (APCs) or cytotoxic T cells that induce immune response is very small or the cold tumor environment where the function of immune cells is inhibited by various Immunosuppressive factors. Therefore, many researches are focusing on turning cold tumor which is unresponsive to immune anticancer agents into hot tumor in order to increase the response rate for immunotherapy.

Among them, induction of immunogenic cell death (ICD), which can activate immune cells and induce the infiltration of immune cells into tumors, is drawing attention. During the process where cancer cells are killed by immunogenic cell death, immunogenic materials such as tumor antigens and damage-associated molecular patterns (DAMPs) are released. These immunogenic materials stimulate immune cells and induce antitumor immune response by recruiting cytotoxic T cells to the tumor site. Accordingly, a combination therapy with a chemo-anticancer agent capable of inducing immunogenic cell death has been presented as a strategy for reversing multiple immunosuppressive activity of the tumor microenvironment in cancer showing a low response rate for the existing immune anticancer therapy and increasing anticancer immune response.

Until now, various drug carrier technologies have been developed for co-delivery of multiple drugs to tumors. However, most of them depend only on EPR (enhanced permeation and retention) without a targeting ligand. Accordingly, there are disadvantages that, in addition to the risk of side effects of nonspecific drug delivery to normal tissues, the therapeutic effect is decreased due to the lack of delivery specificity to cancer cells at the tumor site where various types of cells are present. In particular, also in the immune anticancer therapy that utilizes the immune system of the human body, there is the problem that various side effects occur due to the immune response to normal tissues.

### DISCLOSURE

### Technical Problem

The present disclosure is directed to providing a liposome-based drug carrier for immune anticancer therapy for inducing a continuous tumor-specific anticancer immune response in cancer patients having a low response rate to a standalone immunogenic anticancer agent therapy, and to reducing side effects due to non-specific drug delivery, thereby increasing the effects of immune anticancer therapy, and a method for preparing the same.

### Technical Solution

According to an aspect of the present disclosure, there is provided a liposome-based drug carrier for immune anticancer therapy, which includes: a liposome; an aptamer which is a target-specific ligand for cancer cells conjugated on the surface of the liposome; and an immunogenic chemo-anticancer agent and siRNA, an immunosuppressive protein expression inhibitor, which are co-loaded to the liposome.

The aptamer may be an aptamer for any cancer cell biomarker selected from HER3, NF-kB, PSMA, CD137, PD-1, PD-L1, CD134, PDGF, VEGF, NCL, β-catenin, CD44, CD276, CD133, EGFR, CXCR4, COPS2, VCAN, TNC, THBS2, SRRT, DNAJA1, DPYSL2, AHCY, PGK1, EHD2, ADH1B, ALK, KRAS, ROS1, BRAF, NTRK, MET, RET, MUC1 and HER2.

The aptamer may further include an aptamer for any cancer cell immune checkpoint protein selected from PD-1, PD-L1, PD-L2 and 4-1BB, such that the two aptamers are co-conjugated on the surface of the liposome.

The aptamer for the cancer cell immune checkpoint protein may act as an antagonist of the cancer cell immune checkpoint protein.

The aptamer may be a dual aptamer including a CD44 target-specific ligand and a PD-L1 target-specific ligand.

The immunogenic chemo-anticancer agent may be anyone selected from doxorubicin, oxaliplatin, epirubicin, docetaxel, paclitaxel, valrubicin, cisplatin and tamoxifen.

The immunosuppressive protein expression inhibitor siRNA may be an siRNA that inhibits the production of any protein selected from IDO-1, PD-L1, CTLA4, arginase-1, TGFβ and iNOS for immune evasion of cancer cells.

The cationic lipid may be anyone selected from DOTAP (dioleoyl-3-trimethylammonium propane), DOTMA (1,2-di-O-octadecenyl-3-trimethylammonium-propane), DDAB (dimethyl-dioctadecylammonium bromide) and DODMA (1,2-dioleyloxy-3-dimethylaminopropane).

The cationic lipid and the siRNA may be loaded such that the ratio of the amino group of the cationic lipid and the phosphate group of the siRNA is 1:1 to 6:1.

According to another aspect of the present disclosure, there is provided a method for preparing a liposome-based drug carrier for immune anticancer therapy, which includes:
(a) a step of preparing a cationic liposome containing siRNA, which is an immunosuppressive protein inhibitor, and a micelle;
(b) a step of forming a liposome particle containing the immunosuppressive protein inhibitor siRNA, with a predetermined size, by mixing and sonicating the cationic liposome and the micelle, and then extruding the same;
(c) a step of preparing an aptamer-conjugated micelle by reacting an aptamer which is a cancer cell target-specific ligand with the micelle; and
(d) a step of mixing the aptamer-conjugated micelle, the liposome particle containing the immunosuppressive protein inhibitor siRNA, and an immunogenic chemo-anticancer agent.

In the step (a), the cationic liposome containing the immunosuppressive protein inhibitor siRNA may be prepared by hydrating a dry lipid membrane containing a cationic lipid with a buffer containing the immunosuppressive protein inhibitor siRNA.

The cationic lipid and the siRNA may be used such that the ratio of the amino group of the cationic lipid and the phosphate group of the siRNA is 1:1 to 6:1.

The step (d) may be performed at 50 to 70° C.

According to another aspect of the present disclosure, there is provided an anticancer agent composition containing the liposome-based drug carrier for immune anticancer therapy.

The anticancer agent composition may be for treating any cancer selected from breast cancer, lung cancer, colorectal cancer, stomach cancer, liver cancer, gallbladder cancer, pancreatic cancer, cervical cancer, leukemia, lymphoma and prostate cancer.

### Advantageous Effects

A liposome-based drug carrier for immune anticancer therapy of the present disclosure may be used as a targeted therapeutic agent which specifically co-delivers an immunogenic cell death-inducing chemo-anticancer agent and an immunosuppressive protein inhibitor only to target cancer cells. Of the two aptamers on the surface of the liposome carrier, one is used as a target-specific ligand and the other increases the immune response of cytotoxic T cells to cancer cells by acting as an antagonist for the immune checkpoint protein of the cancer cells. As a result, it is possible to induce a continuous tumor-specific anticancer immune response in cancer patients having a low response rate to a standalone immunogenic anticancer agent therapy, and to reduce side effects due to non-specific drug delivery, thereby remarkably improving therapeutic effect.

### BRIEF DESCRIPTION OF DRAWINGS

FIG. 1 schematically shows the antitumor effect of chemo-anticancer agent/immune anticancer agent (doxorubicin, IDO1 siRNA) co-administration using a liposome-based drug carrier according to an exemplary embodiment of the present disclosure.

FIGS. 2A to 2D show a result of analyzing the efficacy of doxorubicin (DOX) as a potent ICD inducer in a breast cancer 4T1 tumor model and cancer cells in Example 1.

FIG. 3 shows the tumor growth curves of mice vaccinated with PBS-, DOX- or CIS-treated 4T1 cells in Test Example 1.

FIG. 4 shows a result of investigating the IDO1 expression level in tumors obtained by vaccine approach in order to examine the relationship between ICD and IDO1 expression in TME in Test Example 1.

FIGS. 5A and 5B show a result of measuring the optimal N/P ratio for effective siRNA loading to a cationic liposome in Test Example 2.

FIG. 6 shows a result of measuring the fluorescence of a Cy5.5-labeled DNA aptamer in Test Example 2.

FIG. 7 shows the transmission electron microscopy (TEM) images and diameter distribution of Lipm [DOX/IDO1] and Aptm [DOX/IDO1] in Test Example 2.

FIG. 8 shows a result of measuring the zeta potential of Lipm [DOX/IDO1] and Aptm [DOX/IDO1] in Test Example 2.

FIG. 9 shows the fluorescence microscopic images for investigating the cell-specific binding of a CD44/PD-L1 aptamer-conjugated liposome for CD44/PD-L1-positive breast cancer cells (MDA-MB-231 and 4T1) in Test Example 3.

FIG. 10 shows a result of measuring the aptamer-mediated internalization of Aptm [DOX/IDO1] in Test Example 3.

FIG. 11 shows a result of investigating the intracellular distribution of DOX and IDO1 siRNA delivered by a CD44/PD-L1 aptamosome in Test Example 4.

FIG. 12 shows a result of identifying the presence of FITC-labeled IDO1 siRNA in the cytoplasm for Lipm [DOX/IDO1] and Aptm [DOX/IDO1] in Test Example 4.

FIG. 13 shows a result of analyzing the apoptotic cell death of breast cancer cells treated with free DOX, Lipm [DOX/IDO1] and Aptm [DOX/IDO1] by flow cytometry in Test Example 5.

FIG. 14 shows a result of investigating extracellular ATP release and the expression of CRT, an ICD marker, on cell surface in DOX-induced ICD in Test Example 5.

FIG. 15 shows a result of analyzing the inhibition of IDO1 expression in breast cancers in Test Example 5.

FIG. 16 shows a result of analyzing cytotoxicity in Test Example 5.

FIG. 17 shows a result of monitoring the distribution of DOX by measuring the fluorescence of DOX by IVIS imaging in Test Example 6.

FIG. 18 shows a result of investigating the anticancer efficacy of Aptm [DOX/IDO1] by measuring tumor size in Test Example 6.

FIG. 19 shows a result of analyzing antitumor effect by investigating the ratio of CTLs (CD8α+ in mouse T cells), T_{reg}s (FoxP3+ in CD4+ T cells) and mature dendritic cells (DCs) in 4T1 tumor xenograft mice by flow cytometry in Test Example 7.

FIG. 20 shows a result of analyzing the expression level of IDO1 in tumor tissues in Test Example 7.

FIG. 21 shows a result of investigating whether the administration of Aptm [DOX/IDO1] can inhibit tumor metastasis in 4T1 tumor-bearing mice in Test Example 7.

### BEST MODE

Hereinafter, several aspects and various exemplary embodiments of the present disclosure are described more specifically. Hereinafter, the exemplary embodiments of the present disclosure will be described in detail referring to the attached drawings so that those having ordinary knowledge in the art to which the present disclosure belongs can easily carry out the present disclosure. However, the following description is not intended to limit the present disclosure to specific exemplary embodiments. In describing the present disclosure, when it is determined that a detailed description of a related known technology may obscure the gist of the present disclosure, the detailed description will be omitted. The terminology used in herein is used only to describe specific exemplary embodiments, and is not intended to limit the present disclosure. The singular expression includes the plural expression unless the context clearly dictates otherwise. In the present disclosure, terms such as “include”, “have”, etc. are intended to designate that a feature, number, step, operation, element, or combination thereof described in the specification exists, and they not should be understood as precluding in advance the possibility of the presence or addition of one or more other features, numbers, steps, operations, elements, or combinations thereof.

Hereinafter, a liposome-based drug carrier for immune anticancer therapy of the present disclosure is described.

The liposome-based drug carrier for immune anticancer therapy of the present disclosure includes: a liposome; an aptamer which is a target-specific ligand for cancer cells conjugated on the surface of the liposome; and an immunogenic chemo-anticancer agent and siRNA, an immunosuppressive protein expression inhibitor, which are co-loaded to the liposome.

The aptamer may be a target-specific aptamer for any cancer cell biomarker selected from HER3, NF-kB, PSMA, PD-1, PD-L1, CD137, CD134, PDGF, VEGF, NCL, β-catenin, CD44, CD276, CD133, EGFR, CXCR4, COPS2, VCAN, TNC, THBS2, SRRT, DNAJA1, DPYSL2, AHCY, PGK1, EHD2, ADH1B, ALK, KRAS, ROS1, BRAF, NTRK, MET, RET, MUC1 and HER2.

Specifically, the aptamer may further include an aptamer for any cancer cell immune checkpoint protein selected from PD-1, PD-L1, PD-L2 and 4-1 BB, such that the two aptamers are co-conjugated on the surface of the liposome.

The aptamer for the cancer cell immune checkpoint protein may increase the immune response of cytotoxic T cells to cancer cells by acting as an antagonist of the cancer cell immune checkpoint protein.

More specifically, the aptamer may be a dual aptamer including a CD44 target-specific ligand and a PD-L1 target-specific ligand.

The immunogenic chemo-anticancer agent may be selected from doxorubicin, oxaliplatin, epirubicin, docetaxel, paclitaxel, valrubicin, cisplatin, tamoxifen, etc.

The immunosuppressive protein expression inhibitor siRNA may be an siRNA that inhibits the production of any protein selected from IDO-1, PD-L1, CTLA4, arginase-1, TGFβ and iNOS for immune evasion of cancer cells.

The liposome may include a cationic lipid, a non-cationic lipid and a bilayer stabilizer.

The non-cationic lipid may be dioleoylphosphatidylethanolamine (DOPE), dipalmitoylphosphatidylcholine (DPPC), palmitoyloleoylphosphatidylcholine (POPC), egg phosphatidylcholine (EPC), distearoylphosphatidylcholine (DSPC), cholesterol, etc., specifically dioleoylphosphatidylethanolamine (DOPE) or cholesterol.

The bilayer stabilizer may be a polyethylene glycol (PEG) derivative. By introducing the polyethylene glycol (PEG) derivative to the liposome, the blood residence time of the liposome drug carrier may be extended and the aptamer may be modified. Specifically, the polyethylene glycol (PEG) derivative may be DSPE-PEG-maleimide {1,2-distearoyl-sn-glycero-3-phosphoethanolamine-N-[maleimide(polyethylene glycol)]}.

The cationic lipid may be DOTAP (dioleoyl-3-trimethylammonium propane), DOTMA (1,2-di-O-octadecenyl-3-trimethylammonium-propane), DDAB (dimethyl-dioctadecylammonium bromide), DODMA (1,2-dioleyloxy-3-dimethylaminopropane), etc., specifically DOTAP (dioleoyl-3-trimethylammonium propane).

Most specifically, the liposome may include DOPE (1,2-dioleoyl-sn-glycero-3-phosphatidylethanolamine), DOTAP (1,2-dioleoyl-3-trimethylammonium-propane), cholesterol and DSPE-PEG-maleimide {1,2-distearoyl-sn-glycero-3-phosphoethanolamine-N-[maleimide(polyethylene glycol)]}.

The cationic lipid and the siRNA may be used such that the ratio of the amino group of the cationic lipid and the phosphate group of the siRNA is 1:1 to 6:1.

The cationic lipid and the siRNA may be loaded at a specific ratio owing to the electrostatic reaction between the cationic lipid and the siRNA.

Hereinafter, a method for preparing a liposome-based drug carrier for immune anticancer therapy of the present disclosure will be described.

First, a cationic liposome containing siRNA, which is an immunosuppressive protein inhibitor, and a micelle are prepared (step a).

The cationic liposome containing the immunosuppressive protein inhibitor siRNA may be prepared by hydrating a dry lipid membrane containing a cationic lipid with a buffer containing the immunosuppressive protein inhibitor siRNA.

The cationic lipid is the same as described above and, when it is DOTAP, DOTAP and siRNA may be used such that the ratio of the arginine amino group of the DOTAP and the phosphate group of the siRNA is 1:1 to 6:1.

Next, a liposome particle containing the immunosuppressive protein inhibitor siRNA, with a predetermined size, is formed by mixing and sonicating the cationic liposome and the micelle, and then extruding the same (step b).

Then, an aptamer-conjugated micelle is prepared by reacting an aptamer which is a cancer cell target-specific ligand with the micelle (step c).

Finally, the aptamer-conjugated micelle, the liposome particle containing the immunosuppressive protein inhibitor siRNA, and an immunogenic chemo-anticancer agent are mixed (step d).

Specifically, this step may be performed at 50 to 70° C.

The immunogenic chemo-anticancer agent may be encapsulated in the liposome via a pH gradient inside and outside the liposome.

Through this, an aptamer-conjugated liposome-based targeted drug carrier with a diameter of 160 to 200 nm is formed finally.

The present disclosure also provides an anticancer agent composition containing the liposome-based drug carrier for immune anticancer therapy.

The anticancer agent composition may be for treating cancer such as breast cancer, lung cancer, colorectal cancer, stomach cancer, liver cancer, gallbladder cancer, pancreatic cancer, cervical cancer, leukemia, lymphoma, prostate cancer, etc.

### MODE FOR INVENTION

Hereinafter, the present disclosure will be described specifically through examples.

### EXAMPLE

### Example: Preparation of DNA Aptamer-Conjugated Liposome Containing Doxorubicin (DOX) and IDO1 siRNA

(1) Preparation of Cationic Liposome Containing Micelle and IDO1 siRNA

Specifically, a lipid mixture for preparation of a liposome was prepared by mixing cholesterol, DOTAP (1,2-dioleoyl-3-trimethylammonium-propane), DSPE-PEG2000 and DOPE (1,2-dioleoyl-sn-glycero-3-phosphatidylethanol-amine) at a molar ratio of 50:50:1:1 in a mixture of chloroform and methanol (2:1, v/v).

A micelle (0.3 mg) was formed by mixing DSPE-PEG2000 and DSPE-PEG2000-maleimide at a molar ratio of 1:4 in a mixture of chloroform and methanol (2:1, v/v).

In order to evaporate the chloroform and methanol used for preparation of the liposome and micelle, the mixture was evaporated completely for at least 1 hour using N₂ stream and vacuum drying.

A cationic liposome was prepared by hydrating a dried lipid membrane with 1 mL of a citrate buffer (20 mM citric acid and 150 mM NaCl, pH 4.0) containing IDO1 siRNA (N/P ratio of DOTAP and siRNA=4:1, N=arginine amino groups of cationic lipid DOTAP, P=phosphate groups of siRNA) and 200 U of an RNase inhibitor and 0.3 mL of a HEPES buffer (20 mM HEPES and 150 mM NaCl, pH 7.4).

After the hydration, the liposome and the micelle were sonicated for 5 minutes while vortexing with a bath-type sonicator with 1-minute intervals. Next, the size of the liposome was decreased gradually by a liposome solution sequentially through 400, 200 and 100 nm polycarbonate membranes. The buffer solution containing the cationic liposome was exchanged with a HEPES buffer (pH 7.4) by centrifuging at 1600 g for 1 hour using Centricon (30 kDa MWCO, Millipore).

### (2) Preparation of DNA-Aptamer-Conjugated Micelle

For conjugation with the micelle and an aptamer, a DNA oligopolynucleotide including a CD44- or PD-L1-specific aptamer sequence and an additional 3-terminal sequence was synthesized, and the 5′- and 3-terminals were modified with FAM (6-fluorescein amidite) or a Cy5.5 fluorophore, and a thiol, respectively.

The sequence of the anti-CD44 aptamer was 5′-CCAAGGCCTGCAAGGGAACCAAGGACACAGTTTTTTTTTT-3′, and the sequence of anti-PD-L1 was 5′-ACGGGCCACATCAACTCATTGATAGACAATGCGTCCACTGCCCGTTTTTTTTTT-3′.

Prior to the aptamer-micelle conjugation, 1 nmol of each aptamer or fluorophore-labeled aptamer was incubated with 10 μL of TCEP [tris(2-carboxyethyl) phosphine, 100 mM] at 25° C. for at least 30 minutes, and then purified with 3 M NaOAc and cooled ethanol.

Monoaptamer-conjugated micelles were prepared by conjugating the two purified DNA aptamers (1 nmol) to the maleimide groups of the micelle (respectively 0.15 mg) at 25° C. for at least 2 hours.

(3) Post-Insertion of Aptamer-Conjugated Micelle and Encapsulation of DOX in Cationic Liposome Containing siRNA

Aptm [DOX/IDO1] was formed by post-insertion and pH gradient-based methods. The two aptamer-micelle conjugates were mixed with the liposome and 60 μL of DOX (5 mg/mL) and incubated at 60° C. for 1 hour. During this process, the aptamer-micelle conjugate was inserted into the liposome with a loose structure at 60° C., and DOX was loaded to the liposome at the same time due to the difference in pH inside and outside the liposome.

For comparison, an aptamer-free liposome containing DOX and IDO1 siRNA (Lipm [DOX/IDO1]) was prepared using a micelle not conjugated to an aptamer. The free aptamer and DOX were removed from both the Lipm [DOX/IDO1] and Aptm [DOX/IDO1] by CL4B size-exclusion chromatography. The eluted fraction containing Aptm [DOX/IDO1] was concentrated by centrifuging at 1600×g for 20 minutes. For quantification of the DOX and IDO1 siRNA loaded to the liposome, 54 μL of Lipm [DOX/IDO1] or Aptm [DOX/IDO1] was mixed with 6 μL of 10% Triton X-100 and heated at 95° C. for 5 minutes to disrupt the structure of the liposome.

50 μL of the sample solution was transferred to each well of a 96-well plate, and the released DOX and IDO1 siRNA were analyzed by measuring fluorescence using a microplate reader (λₑₓ=485 nm and λₑₘ=670 nm for DOX; λₑₓ=485 nm and λₑₘ=535 nm for FITC-labeled IDO1 siRNA).

Standard curves were plotted using sequentially diluted DOX or FITC-labeled siRNA supplemented with 1% Triton X-100. Then, the amount of DOX and IDO1 siRNA loaded to Aptm [DOX/IDO1] was determined from the standard curves.

DOX loading efficiency (EDOX) and IDO1 siRNA loading efficiency (EIDO1 siRNA) were calculated from the following equations.

EDOX=100×[DOX concentration in Aptm [DOX/IDO1 (μg/mL)]×solution volume (mL)]/DOX amount μg) input; EIDO1 siRNA=100×[siRNA concentration in Aptm [DOX/IDO1 (μg/mL)]×solution volume (mL)]×solution volume]/siRNA amount (μg) input.

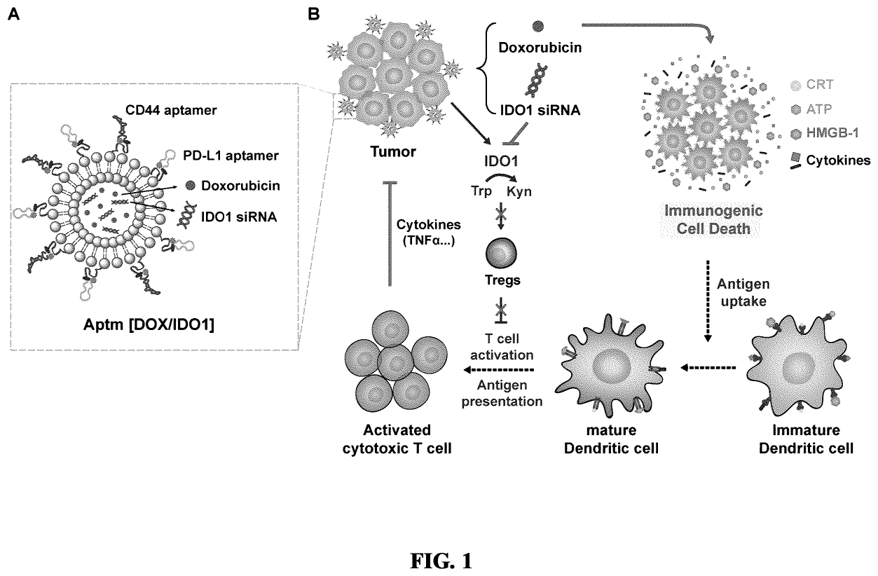

FIG. 1 schematically shows the antitumor effect of chemo-anticancer agent/immune anticancer agent (doxorubicin, IDO1 siRNA) co-administration using the liposome-based drug carrier according to an exemplary embodiment of the present disclosure. The two DNA aptamers specifically binding to membrane proteins (CD44, PD-L1) overexpressed on the surface of cancer cells were modified with a fluorophore at the 5′-end, and the 3′-end was modified with a thiol functional group (—SH) for function as a ligand for targeted delivery on the surface of the liposome carrier through covalent bonding with the maleimide functional group of the liposome. Among them, the PD-L1 aptamer can inhibit the action of killer T cells for cancer cells by acting as an antagonist for the immune checkpoint protein PD-L1 of cancer cells.

### TEST EXAMPLES

### Test Example 1: Confirmation of DOX-Induced ICD and IDO1 Upregulation

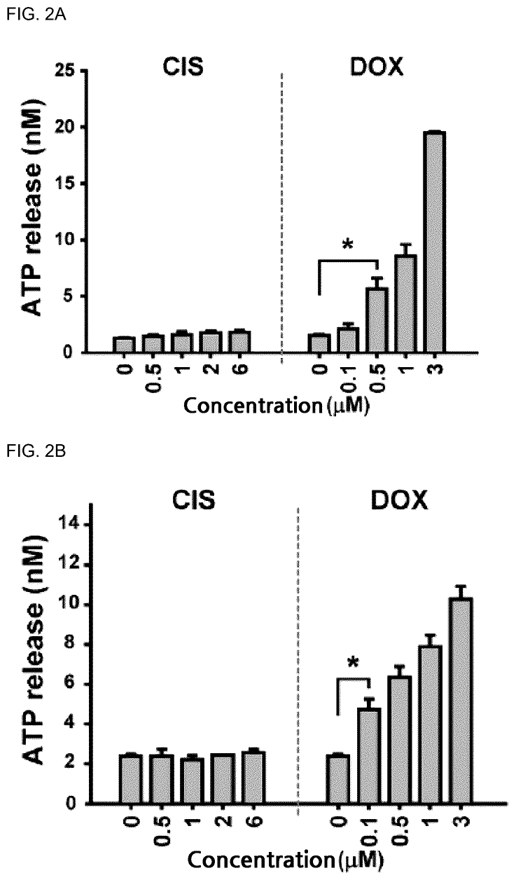

First, the efficacy of doxorubicin (DOX) as a potent ICD inducer in a breast cancer 4T1 tumor model and cancer cells was tested by monitoring the change in the TME (tumor microenvironment). To validate the immunogenic effect of DOX, immunogenic cell death (ICD) biomarkers, such as extracellularly released ATP and cell surface expressing CRT, were monitored in breast cancer cells (human: MDA-MB-231 cells; mouse: 4T1 cells) treated with different concentrations of DOX and cisplatin (CIS), a non-ICD inducer, as a comparative control group. The result is shown in FIGS. 2A to 2D. Both in the human and mouse breast cancer cells, the level of extracellularly released ATP increased gradually with the concentration of DOX. However, the treatment with CIS did not increase the level of extracellular ATP (FIGS. 2A and 2B). Whereas the CRT expression on the cancer cell surface increased gradually as the concentration of DOX increased, the treatment with CIS did not increase the CRT expression on the cancer cell surface (FIGS. 2C and 2D).

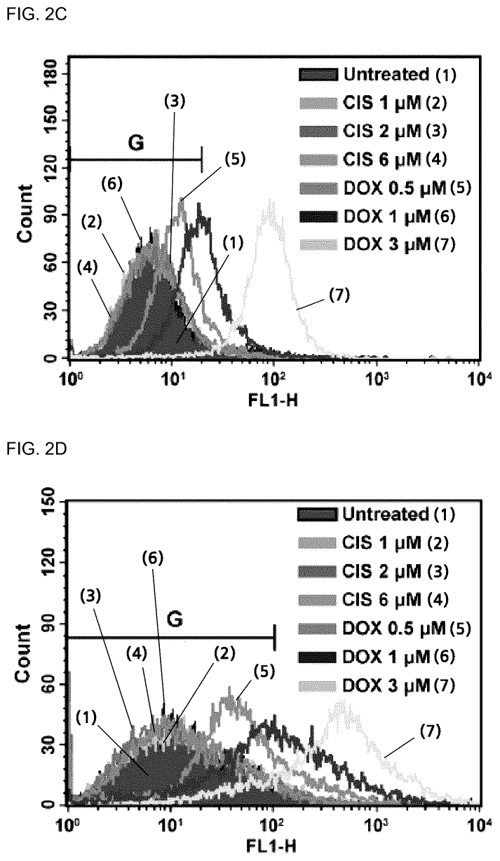

The effect of DOX-induced ICD in Balb/c mice was further validated by vaccine approach. Dead 4T1 cells were subcutaneously injected into one flank of Balb/c mice together with an anticancer agent such as DOX (30 μM) or CIS (100 μM), or phosphate-buffered saline (PBS; control), twice for 2 weeks, with a 1-week interval. At two weeks after the first injection, live 4T1 cancer cells were injected into the opposite flank and the change in tumor size was evaluated for 25 days. FIG. 3 shows the tumor growth curves of the mice vaccinated with PBS-, DOX- or CIS-treated 4T1 cells.

Whereas the vaccination with DOX-treated dead 4T1 cells effectively inhibited tumor growth at the infection site, the PBS- and CIS-treated cells did not inhibit tumor growth significantly. ICD-mediated immune response can invalidate the immune evasion mechanism of TME, such as overexpression of the immune checkpoint protein and IDO1. Accordingly, the expression of IDO1 and PD-L1 under IFNγ stimulation was monitored also in MDA-MB-231 and 4T1 cells.

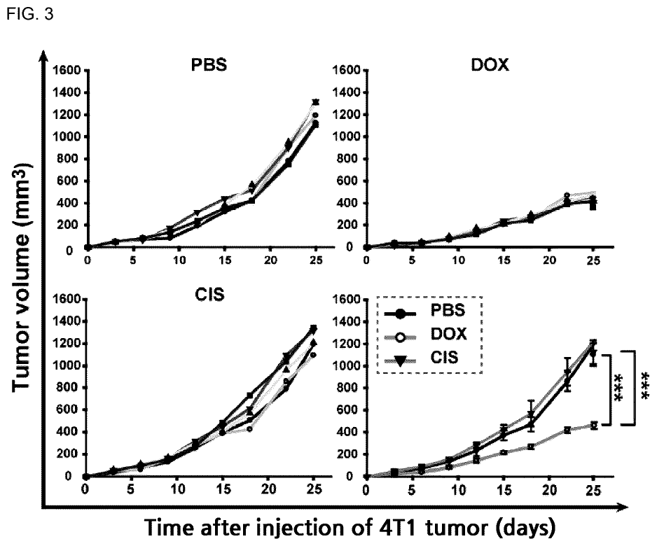

In the present disclosure, the IFNγ stimulation significantly upregulated the expression level of IDO1 in both cell types. PD-L1 was highly expressed also on the surface of the IFNγ-treated cells. Subsequently, it was investigated whether the DOX-induced ICD can induce the expression of IDO1 in a breast cancer mouse model as an indicator of the immune evasion mechanism in TME. In order to investigate the relationship between the expression of ICD and IDO1 in TME, the expression level of IDO1 in the tumors obtained by vaccine approach was investigated, and the result is shown in FIG. 4. The mice vaccinated with the DOX-treated 4T1 cells showed the highest IDO1 protein and mRNA expression in tumor tissues. The mice vaccinated with PBS- or CIS-treated 4T1 cells showed increased expression of IDO1 protein and mRNA as compared to the control group 4T1 cells due to the innate immune response induced by the injected cancer cells. Accordingly, it was confirmed that the vaccination using DOX-treated cells can significantly inhibit tumor growth through ICD-based anticancer immune response. Meanwhile, it was confirmed that the DOX-induced ICD upregulates IDO1 expression in tumor tissues via the immune evasion mechanism.

Therefore, it can be seen that a combination therapy including both the inhibition of IDO1 and an effective ICD-inducing chemotherapeutic agent is necessary.

### Test Example 2: Synthesis of Liposome Carrier for Co-Delivery of DOX and IDO1 siRNA

For co-delivery of DOX and IDO1 siRNA to breast cancer cells, a liposome carrier was constructed with a surface-conjugated functional DNA aptamer.

For targeted co-delivery of DOX and IDO1 siRNA with the liposome carrier to breast cancer cells, anti-CD44 and anti-PD-L1 DNA aptamers, which are specific for the breast cancer cell surface markers CD44 and PD-L1, were conjugated to a liposome. Especially, the antagonistic PD-L1 DNA aptamer can inhibit PD-1/PD-L1 interaction between cancer cell and cytotoxic T lymphocytes (CTLs) by blocking PD-L1. The multimodal cancer cell-targeting DNA aptamer-conjugated liposome for ICD induction and siRNA co-delivery was named Aptm [DOX/IDO1]. First, the 5′- and 3′-ends of the anti-CD44 and anti-PD-L1 DNA aptamers including oligo-T linkers (10 polynucleotides) were modified with a fluorescent dye and a thiol group, respectively.

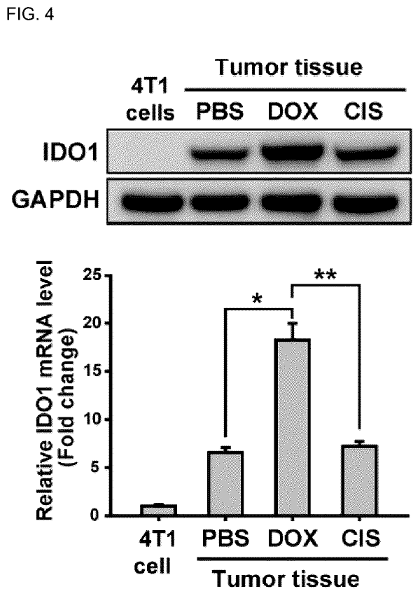

The thiol-modified CD44 and PD-L1 DNA aptamers were covalently bonded to the maleimide group of a PEGylated DSPE micelle, respectively, by a thiol-maleimide bond. Next, the optimal N/P ratio (N=arginine amino group of cationic lipid, DOTAP; P=phosphate group of siRNA) for effective siRNA loading to the cationic liposome was determined, and the result is shown in FIGS. 5A and 5B. After mixing lipids according to the appropriate composition of the cationic liposome, dry lipid films containing different amounts of lipids were hydrated with citrate buffer solutions containing 100 pmol of FITC-labeled siRNA at various N/P ratios (1:1 to 6:1). When the lipid membrane was hydrated with citrate buffers containing siRNA with N/P ratios of 1:1 and 2:1, free siRNA not loaded to the cationic liposome was observed at the bottom of 1% agarose gel. In contrast, most siRNAs were captured in the well of the gel at N/P ratios of 4:1 and 6:1. This indicates that siRNA was effectively loaded in the liposome at high N/P ratios.

Accordingly, the N/P ratio for effective siRNA loading to the cationic liposome was selected as 4:1. Then, a cationic liposome containing IDO1 siRNA was prepared by hydrating a lipid film composed of cholesterol, DOTAP (1,2-dioleoyl-3-trimethylammonium-propane), DSPE-PEG2000 and DOPE (1,2-dioleoyl-sn-glycero-3-phosphatidylethanolamine) with a citrate buffer containing IDO1 siRNA. The cationic liposome containing IDO1 siRNA was then fused with an aptamer-conjugated micelle at high temperature (60° C.) in the presence of an ICD inducer (DOX) using the post-insertion method. DOX was co-loaded with the liposome via proton exchange passing through the membrane due to the pH gradient inside and outside the liposome. Aptm [DOX/IDO1] was purified from free DOX and unbound DNA aptamer by gel permeation chromatography. After quantification of DOX and siRNA loaded to the liposome, the loading efficiency for DOX and IDO1 siRNA was calculated as 15.9% and 72.7%, respectively.

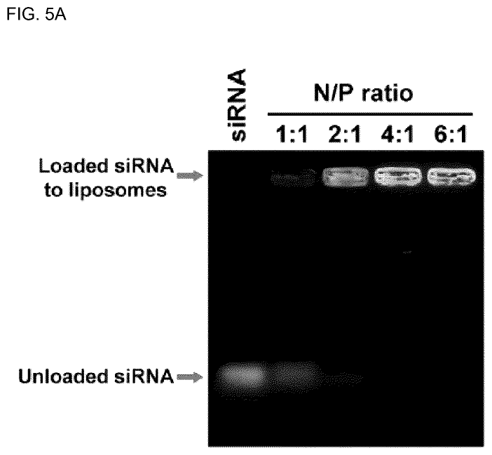

Next, it was confirmed whether the DNA aptamer was conjugated onto the surface of the liposome. After preparing a CD44/PD-L1 aptamer-conjugated liposome (CD44/PD-L1 Aptm) and purifying free nucleic acids by gel permeation chromatography, the fluorescence of the Cy5.5-labeled DNA aptamer was measured. The result is shown in FIG. 6. It was confirmed that CD44/PD-L1 Aptm exhibited much stronger Cy5.5 fluorescence as compared to a normal liposome, indicating that the DNA aptamer was conjugated onto the surface of the liposome. It was also confirmed that the antagonistic PD-L1 aptamer-conjugated liposome (PD-L1 aptamosome and CD44/PD-L1 aptamosome) can inhibit PD-1/PD-L1 binding by blocking PD-L1.

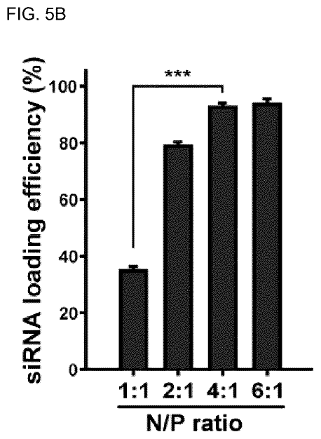

Aptm [DOX/IDO1] was characterized by evaluating the physical properties of Aptm [DOX/IDO1] such as shape, size and zeta potential. FIG. 7 shows the transmission electron microscopy (TEM) images and diameter distribution of Lipm [DOX/IDO1] and Aptm [DOX/IDO1]. It was confirmed that both Lipm [DOX/IDO1] and Aptm [DOX/IDO1], which are liposomes that encapsulate DOX and siRNA, have spherical structures, and the average size of Lipm [DOX/IDO1] and Aptm [DOX/IDO1] was 173 nm and 183 nm, respectively.

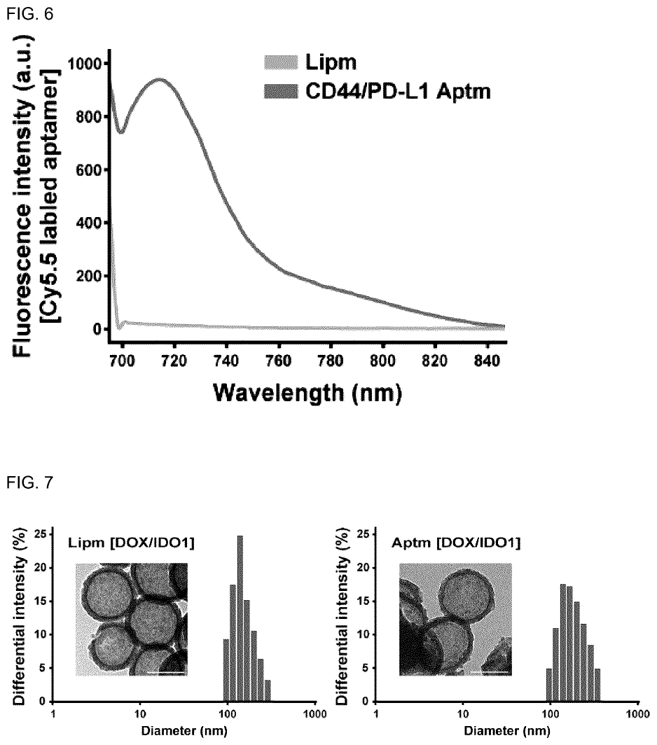

In addition, FIG. 8 shows a result of measuring the zeta potential of Lipm [DOX/IDO1] and Aptm [DOX/IDO1]. The zeta potential of Aptm [DOX/IDO1] was lower than that of Lipm [DOX/IDO1](−1.31 mV vs. 21.8 mV), indicating the presence of the negatively charged DNA aptamer on the surface of Aptm [DOX/IDO1].

### Test Example 3: Confirmation of Aptamer-Mediated Specific Binding and Drug Delivery to Target Cells by Aptm [DOX/IDO1]

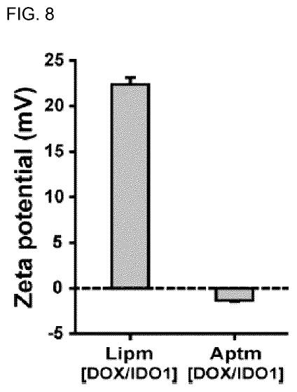

In order to investigate the cell-specific binding of the CD44/PD-L1 aptamer-conjugated liposome for CD44/PD-L1-positive breast cancer cells (MDA-MB-231 and 4T1), the cancer cells were treated with a normal liposome (Lipm). The fluorescence microscopic images of the cells are shown in FIG. 9. CD44 aptamosome (CD44 Aptm), PD-L1 aptamosome (PD-L1 Aptm) or CD44/PD-L1 aptamosome (CD44/PD-L1 Aptm) includes rhodamine-labeled DOPE lipids. The aptamosomes conjugated with the CD44 or PD-L1 aptamer showed increased rhodamine fluorescence around and inside the CD44/PD-L1-positive breast cancer cells as compared to the normal liposome, and the aptamosome conjugated with the two aptamers showed the strongest rhodamine fluorescence. In contrast, CD44/PD-L1-negative HepG2 cells did not show fluorescence that can be detected by the aptamosomes. This indicates that the CD44- and PD-L1-specific DNA aptamers conjugated to the liposome effectively target CD44/PD-L1-positive breast cancer cells.

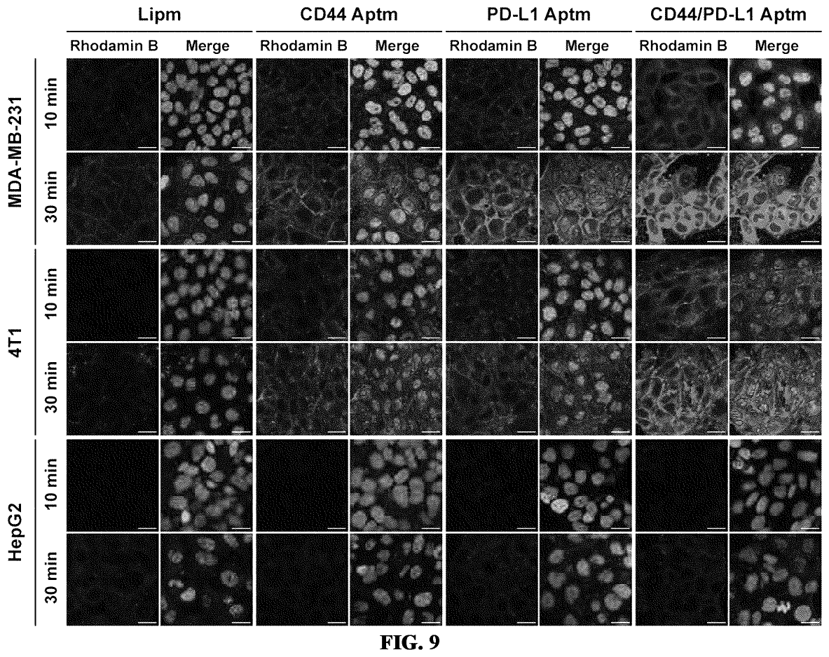

Next, it was evaluated whether Aptm [DOX/IDO1] can deliver a drug to target cancer cells through aptamer-mediated internalization, and the result is shown in FIG. 10. After incubating cells with Lipm [DOX/IDO1], CD44 Aptm [DOX/IDO1], PD-L1 Aptm [DOX/IDO1] and CD44/PD-L1 Aptm [DOX/IDO1] for 40 minutes, the fluorescence by DOX was analyzed by flow cytometry. As in the result of fluorescence microscopic analysis, the dual aptamer-conjugated liposome (CD44/PD-L1 Aptm [DOX/IDO1]) showed the best DOX delivery efficiency with significant migration of the CD44/PD-L1-positive breast cancer cells (orange curve). In contrast, CD44/PD-L1-negative HepG2 cells showed no significant DOX internalization for any aptamer-conjugated liposome.

In order to further identify that the targeted intracellular delivery of DOX by CD44/PD-L1 Aptm [DOX/IDO1] is mediated by the anti-CD44 and anti-PD-L1 DNA aptamers, the target protein was blocked by anti-CD44 and anti-PD-L1 antibodies prior to treatment with CD44/PD-L1 Aptm [DOX/IDO1]. When the target protein was blocked by the antibodies, the DOX delivery efficiency by CD44/PD-L1 Aptm [DOX/IDO1]decreased significantly only in the target cancer cells. Therefore, it was confirmed that the anti-CD44 and anti-PD-L1 aptamer-conjugated liposomes can deliver the drug into CD44/PD-L1 aptamer-positive breast cancer cells through specific binding to the target protein.

### Test Example 4: Confirmation of Intracellular Distribution of DOX and IDO1 siRNA Delivered by Aptm [DOX/IDO1]

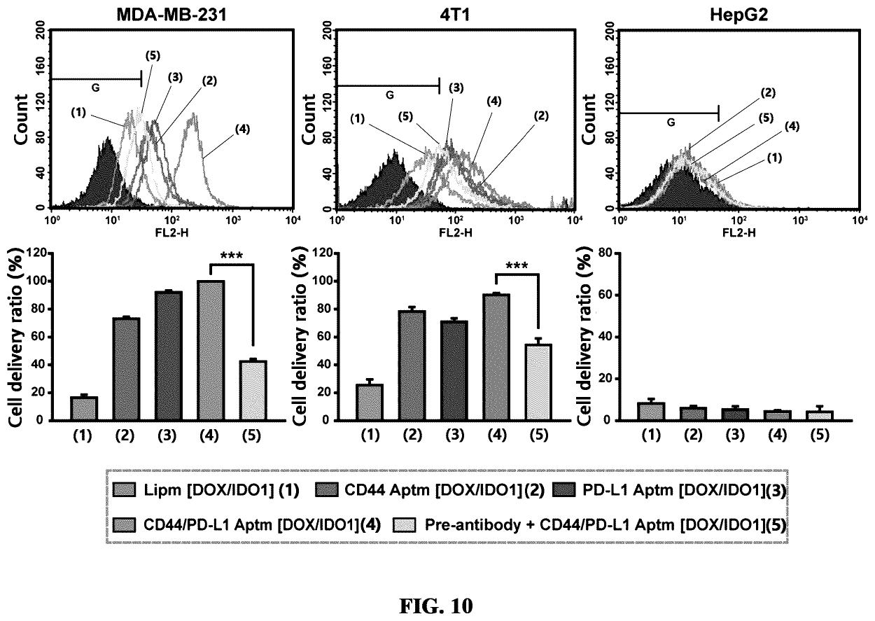

In order to investigate the intracellular distribution of DOX and IDO1 siRNA delivered by the CD44/PD-L1 aptamosome, cells were treated with free DOX, Lipm [DOX/IDO1] and Aptm [DOX/IDO1] and analyzed by confocal microscopy with increasing incubation time. The result is shown in FIG. 11. Whereas the membrane-permeable DOX was accumulated quickly in the nucleus within 1 hour by crossing the membrane, DOX delivered by Lipm [DOX/IDO1] and Aptm [DOX/IDO1] was distributed mainly in the periphery and cytoplasm of the cells up to 1 hour. Nevertheless, a large amount of DOX was delivered easily by Aptm [DOX/IDO1] with the passage of time, and it was gradually delivered into the nucleus of MDA-MB-231 and 4T1 cells within 2 hours. However, most of DOX delivered by Lipm [DOX/IDO1] was still observed in the periphery and cytoplasm of the cells up to 4 hours. When HepG2 cells lacking target surface proteins were treated with Aptm [DOX/IDO1] for 4 hours, DOX delivery was decreased significantly as the cells treated with Lipm [DOX/IDO1].

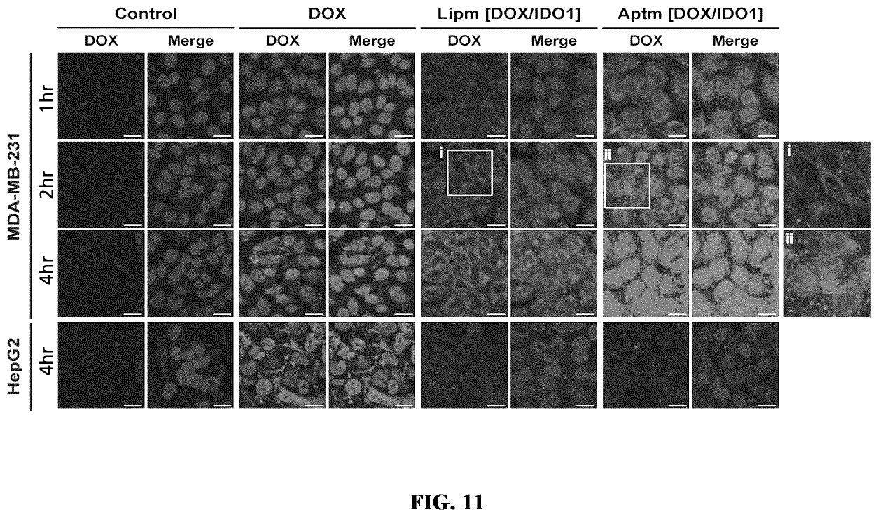

In addition, after treating cells with Lipm [DOX/IDO1] and Aptm [DOX/IDO1] for 12 hours, the presence of FITC-labeled IDO1 siRNA in the cytoplasm was investigated, and the result is shown in FIG. 12. IDO1 siRNA was delivered more effectively to the target breast cancer cells treated with Aptm [DOX/IDO1] than those treated with Lipm [DOX/IDO1]. In addition, Aptm [DOX/IDO1] showed no significant siRNA delivery efficiency in the negative control group HepG2 cells. That is to say, it was confirmed that Aptm [DOX/IDO1] promotes targeted delivery of DOX and IDO1 siRNA to breast cancer cells via the anti-CD44 and anti-PD-L1 aptamers, which are nucleic acid ligands targeting cancer cell surface markers.

### Test Example 5: Analysis of Anticancer Efficacy by Aptm [DOX/IDO1]

Aptm [DOX/IDO1] promotes the delivery of an encapsulated anticancer drug to target breast cancer cells through receptor-mediated endocytosis using a cancer-targeting aptamer. The anticancer effect of a drug and siRNA delivered by Aptm [DOX/IDO1] was analyzed.

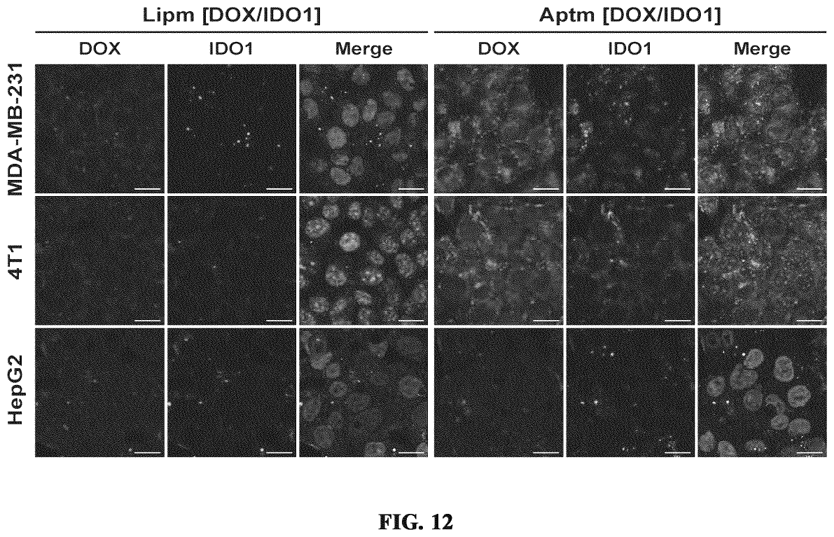

The result of analyzing the apoptotic cell death by an anticancer agent in breast cancer cells treated with free DOX, Lipm [DOX/IDO1] and Aptm [DOX/IDO1] by flow cytometry is shown in FIG. 13. The cell death was increased significantly when the cells were treated with Aptm [DOX/IDO1](11.0±0.38%) as compared to when they were treated with Lipm [DOX/IDO1](4.76±0.38%).

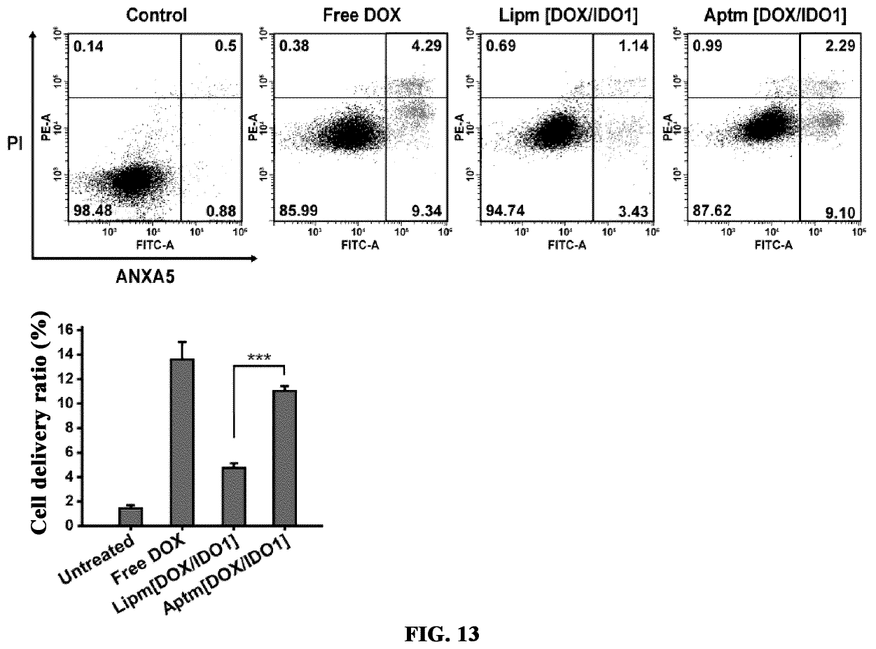

Then, DOX-induced ICD was evaluated by monitoring extracellular ATP release and expression of cell surface CRT, which is an ICD marker, and the result is shown in FIG. 14. A large amount of ATP was released in the breast cancer cells treated with Aptm [DOX/IDO1], similarly to the cells treated with free DOX. In contrast, the group treated with Lipm [DOX/IDO1] released relatively less ATP than the group treated with Aptm [DOX/IDO1]. As the result for ATP release, the CRT expression was also increased significantly on the surface of the cancer cells treated with Aptm [DOX/IDO1] as compared to those treated with Lipm [DOX/IDO1].

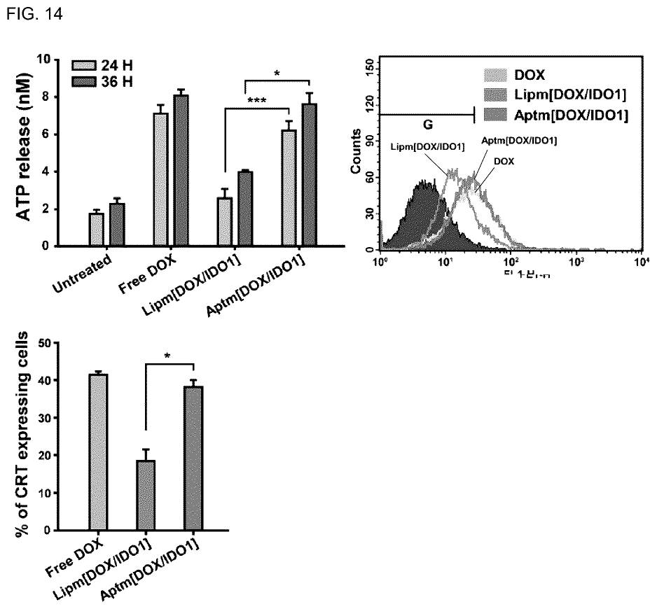

In addition, it was investigated whether Aptm [DOX/IDO1] can inhibit IDO1 expression in breast cancer cells. After inducing IDO1 expression by stimulating breast cancer cells with IFNγ, the cells were treated with Lipm [DOX/IDO1], Aptm [DOX/IDO1], DOX and control group siRNA (Aptm [DOX/Ctr]) for 24 hours, and the degree of inhibition of IDO1 expression was measured. The result is shown in FIG. 15. When the cells were treated with Aptm [DOX/IDO1], the expression of IDO1 protein and mRNA was decreased sufficiently by about 45% and 47%, respectively. In contrast, Lipm [DOX/IDO1] and Aptm [DOX/Ctr] with no cancer cell-targeting aptamer and IDO1 siRNA did not inhibit the expression of IDO1 enough. That is to say, it can be seen that Aptm [DOX/IDO1] significantly increases ICD and downregulates IDO1 expression in target cancer cells through enhanced drug delivery efficiency.

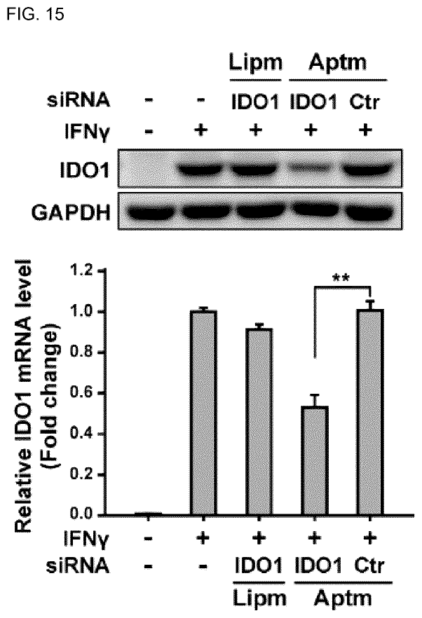

Next, the cytotoxicity by an anticancer agent delivered together with Aptm [DOX/IDO1] was evaluated, and the result is shown in FIG. 16. Breast cancer cells (MDA-MB-231 and 4T1) and control group liver cells (HepG2) were treated with free DOX, Lipm [DOX/IDO1] and Aptm [DOX/IDO1] for 24 hours and 36 hours. Cell survivability was the lowest in the free DOX-treated cells, and the treatment with Aptm [DOX/IDO1] significantly decreased the survivability of the breast cancer cells similarly to free DOX. In addition, whereas the treatment with Aptm [DOX/IDO1] showed stronger toxicity in the breast cancer cells than Lipm [DOX/IDO1], the survivability of the control liver cells (hepatic cells) were similar for the Lipm [DOX/IDO1] and Aptm [DOX/IDO1] treatment.

### Test Example 6: Reduction of Tumor Growth by Biodistribution of DOX and Aptm [DOX/IDO1]

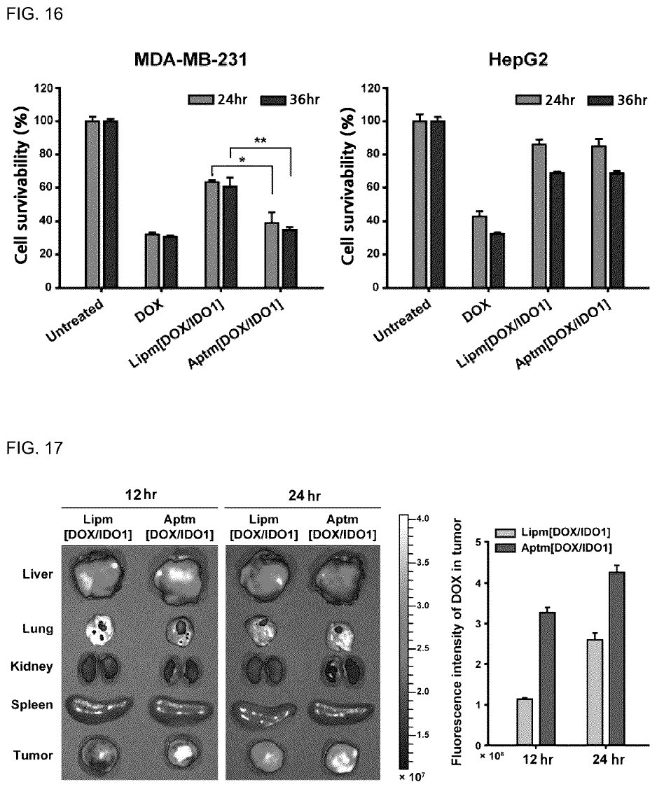

The biodistribution of DOX delivered by Aptm [DOX/IDO1] was evaluated using tumor xenograft Balb/c mice obtained by subcutaneous injection of 4T1 cells. 14 days after the injection of tumor cells, when the tumor volume reached about 400 mm³, Lipm [DOX/IDO1], Aptm [DOX/IDO1] and DOX (6 μg) were injected into the tail vein of the mice. 12 hours and 24 hours after the intravenous injection, the distribution of DOX delivered together with Lipm [DOX/IDO1] or Aptm [DOX/IDO1] to the major organs of the 4T1-tumor-bearing mice was monitored by IVIS imaging, and the result is shown in FIG. 17. When Aptm [DOX/IDO1] was administered to the mice, DOX was accumulated significantly in the tumors. The fluorescence intensity of DOX in the tumors increased by 1.7 times with the passage of time (12 to 24 hours).

The fluorescence of DOX delivered together with Lipm [DOX/IDO1] was lower than that delivered together with Aptm [DOX/IDO1], which is highly likely to be the result of targeting of the nanosized carrier to the tumor site by EPR (enhanced permeation and retention) effect. The biodistribution of DOX shows that the DOX delivered together with Aptm [DOX/IDO1] is accumulated selectively at the tumor site and that Aptm [DOX/IDO1] is a nanocarrier suitable for delivering the anticancer agent to 4T1 tumor xenograft mice.

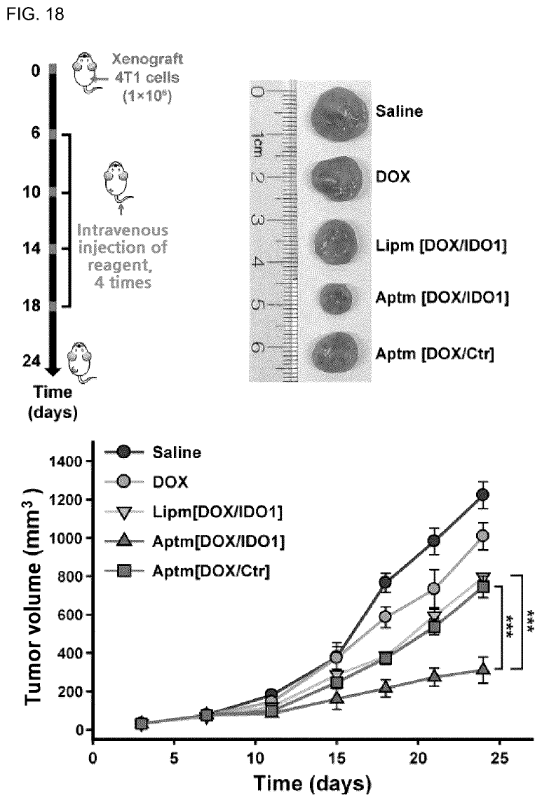

In addition, the anticancer efficacy of Aptm [DOX/IDO1] in the 4T1 tumor xenograft mice was investigated by measuring the decrease of tumor size, and the result is shown in FIG. 18. After the tumor size grew to 60-80 mm³, the mice were divided randomly into 5 groups (5 mice per group) and each reagent was intravenously 4 times with 4-day intervals: saline (control group), free DOX (0.3 mg/kg), Lipm [DOX/IDO1], Aptm [DOX/IDO1] and Aptm [DOX/Ctr], each liposome containing the same amount of DOX (0.3 mg/kg) and siRNA (0.35 mg/kg). The treatment with free DOX did not reduce tumor size enough for 24 days as compared to the treatment with saline. The administration of Lipm [DOX/IDO1] exhibited relatively improved tumor growth with the tumor size decreased by about 33.0% as compared to the administration of saline, which seems to be caused by passive targeting of the liposome for tumor tissues through the EPR effect. The administration of Aptm [DOX/IDO1] inhibited tumor growth the most effectively, with the tumor size decreased by about 71.7% on day 24. Morbidity was not observed when the mice were treated with Aptm [DOX/IDO1]. In particular, when the mice were treated with Aptm [DOX/Ctr] containing scrambled siRNA instead of IDO1 siRNA, the tumor size decreased by about 37.0% (vs. saline administration) due to decreased effect of inhibiting tumor growth. That is, the delivered IDO siRNA inhibits tumor growth by controlling TME. Accordingly, the improved efficiency of reducing tumor growth in the 4T1 tumor xenograft mice by the administration of Aptm [DOX/IDO1] demonstrates that the CD44/PD-L1 aptamosome including an ICD inducer and IDO1 siRNA is delivers the target to the tumor site in vivo in a systematic manner.

### Test Example 7: Confirmation of Control of TME by Antitumor Efficacy

As the effect of Aptm [DOX/IDO1] on the control of tumor microenvironment (TME), cell death, ICD marker, immune cell ratio and TNF secretion in the tumor tissue of 4T1 tumor xenograft mice were evaluated. In order to investigate DOX-induced apoptotic cell death, TUNEL assay was conducted with the tumor tissues of 4T1 tumor xenograft mice. When DOX was delivered together with Aptm [DOX/IDO1] and Aptm [DOX/Ctr], apoptotic cell death increased significantly in the tumor tissue as compared to free DOX or Lipm [DOX/IDO1]. This result indicates that DOX-induced apoptotic cell death improved targeted drug delivery by the CD44/PD-L1 aptamosome via the conjugation with the aptamer.

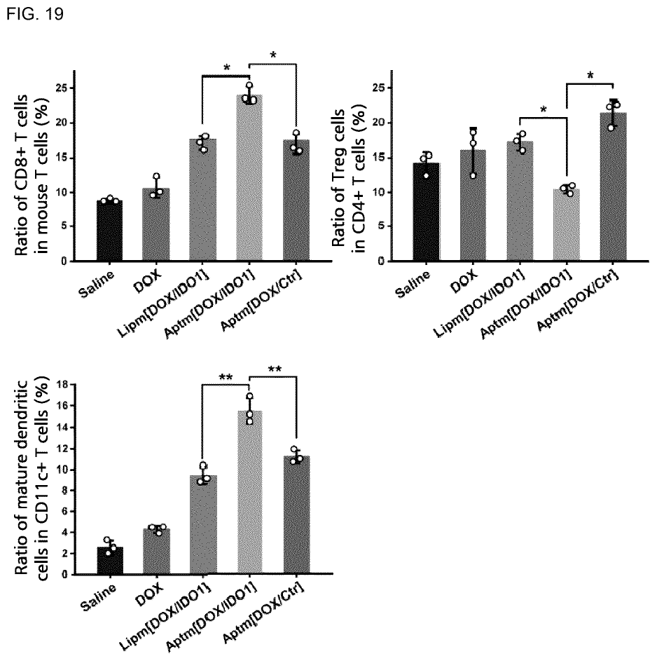

Next, antitumor effect was analyzed by investigating the ratio of CTLs (CD8α+ in mouse T cells), T_{reg}s (FoxP3+ in CD4+ T cells) and mature dendritic cells (DCs) in 4T1 tumor xenograft mice by flow cytometry, and the result is shown in FIG. 19. It was observed that the administration of the aptamer-conjugated liposome in which doxorubicin and IDO1 siRNA were encapsulated together (Aptm [DOX/IDO1]) significantly inhibited tumor growth as compared to the normal liposome not conjugated with the aptamer (Lipm [DOX/IDO1]) or the aptamer-conjugated liposome in which control siRNA was encapsulated (Aptm [DOX/Ctr]). In addition, as a result of investigating the distribution of immune cells in the tumor, it was confirmed that the cytotoxic T cells (CD8+ T cells) and mature dendritic cells associated with antitumor immune response are present at the highest levels and the regulatory T cell (T_{reg}s) that exhibit immunosuppressive activity are present at the lowest level when Aptm [DOX/IDO1] was administered.

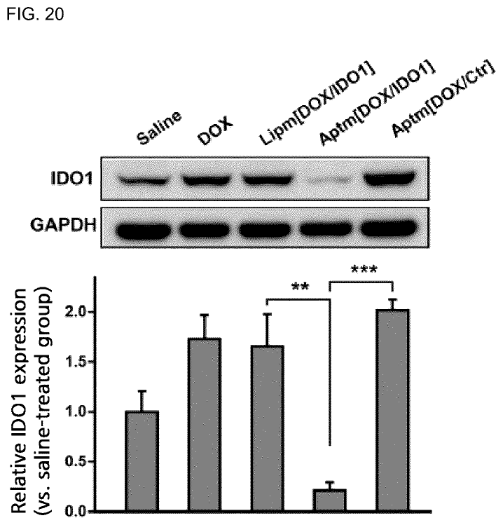

Meanwhile, consistently with the result of enhanced CD8+ CTL infiltration into the tumor, whereas TNFα was released a lot in the tumor of the mice treated with Aptm [DOX/IDO1], relatively low TNFα secretion was observed in the groups treated with free DOX, Lipm [DOX/IDO1] and Aptm [DOX/Ctr]. This result suggests that CD8+ CTLs exhibit antitumor efficacy by secreting TNFα to TME by Aptm [DOX/IDO1]. The effective inhibition of tumor growth improves anticancer immune response by reversing immunosuppressive TME to immunoresponsive TME in vivo by Aptm [DOX/IDO1]. In order to investigate whether tumor size reduction and TME control are mediated by IDO1 siRNA delivered to the 4T1 tumor xenograft mice, the IDO1 expression level in the tumor tissue of the mice was analyzed on day 24, and the result is shown in FIG. 20. The IDO1 expression level in the tumor obtained from the mice treated with free DOX was increased as compared to the IDO1 expression level in the tumor obtained from the mice treated with saline. The treatment with Aptm [DOX/IDO1] significantly decreased IDO1 expression in the tumor as compared to the treatment with Lipm [DOX/IDO1]. In the tumor, the IDO1 expression was increased by the administration of Aptm [DOX/Ctr] lacking IDO1 siRNA. It is thought that the increased IDO1 expression is due to the improved ICD against the tumor due to the effective delivery of DOX using the CD44/PD-L1 aptamosome. Accordingly, the inhibition of IDO1 in combination with ICD can induce anticancer immune response in the immunoresponsive TME by Aptm [DOX/IDO1].

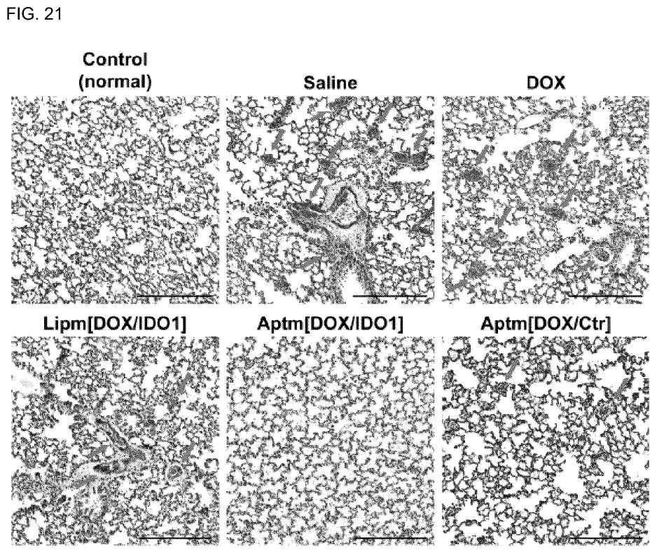

Finally, it was investigated whether the administration of Aptm [DOX/IDO1] can inhibit tumor metastasis in 4T1 tumor-bearing mice. For detection of metastatic tumors, tumor tissues from the terminal part of the lung of the mice were stained with hematoxylin and eosin (H&E), and the images are shown in FIG. 21. When free DOX or Lipm [DOX/IDO1] was administered to the mice, extensive filtration of 4T1 breast cancer cells was observed in the lung, similarly to the tissue of the control group (saline-treated group). In contrast, metastatic tumor was not observed in the lung of the mice treated with Aptm [DOX/IDO1], similarly to the tissue observed from the normal lung. Therefore, it was confirmed that the systemic administration of Aptm [DOX/IDO1] through the blood can inhibit tumor metastasis through targeted delivery of an anticancer drug targeting breast cancer cells.

Although the exemplary embodiments of the present disclosure were described above, those having ordinary knowledge in the art may make various modifications and changes to the present disclosure through addition, change, deletion, etc. of elements without departing from the scope of the present disclosure described in the claims, which are also encompassed within the scope of the present disclosure.

### INDUSTRIAL APPLICABILITY

A liposome-based drug carrier for immune anticancer therapy of the present disclosure can be used as a targeted therapeutic agent which co-delivers an immunogenic cell death-inducing chemo-anticancer agent and an immunosuppressive protein inhibitor specifically only to target cancer cells. Of two aptamers on the surface of the liposome carrier, one is used as a target-specific ligand and the other increases the immune response of cytotoxic T cells to cancer cells by acting as an antagonist for the immune checkpoint protein of the cancer cells. As a result, it is possible to induce a continuous tumor-specific anticancer immune response in cancer patients having a low response rate to a standalone immunogenic anticancer agent therapy, and to reduce side effects due to non-specific drug delivery, thereby remarkably improving therapeutic effect.

We claim:

1. A liposome-based drug carrier for immune anticancer therapy, comprising:
a liposome;
an aptamer which is a target-specific ligand for cancer cells conjugated on the surface of the liposome; and
an immunogenic chemo-anticancer agent and siRNA, an immunosuppressive protein expression inhibitor, which are co-loaded to the liposome.

2. The liposome-based drug carrier for immune anticancer therapy according to claim 1, wherein the aptamer is an aptamer for any cancer cell biomarker selected from HER3, NF-kB, PSMA, PD-1, PD-L1, CD137, CD134, PDGF, VEGF, NCL, β-catenin, CD44, CD276, CD133, EGFR, CXCR4, COPS2, VCAN, TNC, THBS2, SRRT, DNAJA1, DPYSL2, AHCY, PGK1, EHD2, ADH1B, ALK, KRAS, ROS1, BRAF, NTRK, MET, RET, MUC1 and HER2.

3. The liposome-based drug carrier for immune anticancer therapy according to claim 2, wherein the aptamer further comprises an aptamer for any cancer cell immune checkpoint protein selected from PD-1, PD-L1, PD-L2 and 4-1BB, such that the two aptamers are co-conjugated on the surface of the liposome.

4. The liposome-based drug carrier for immune anticancer therapy according to claim 3, wherein the aptamer for the cancer cell immune checkpoint protein acts as an antagonist of the cancer cell immune checkpoint protein.

5. The liposome-based drug carrier for immune anticancer therapy according to claim 3, wherein the aptamer is a dual aptamer comprising a CD44 target-specific ligand and a PD-L1 target-specific ligand.

6. The liposome-based drug carrier for immune anticancer therapy according to claim 1, wherein the immunogenic chemo-anticancer agent is anyone selected from doxorubicin, oxaliplatin, epirubicin, docetaxel, paclitaxel, valrubicin, cisplatin and tamoxifen.

7. The liposome-based drug carrier for immune anticancer therapy according to claim 1, wherein the immunosuppressive protein expression inhibitor siRNA is an siRNA that inhibits the production of any protein selected from IDO-1, PD-L1, CTLA4, arginase-1, TGFβ and iNOS for immune evasion of cancer cells.

8. The liposome-based drug carrier for immune anticancer therapy according to claim 1, wherein the cationic lipid is anyone selected from DOTAP (dioleoyl-3-trimethylammonium propane), DOTMA (1,2-di-O-octadecenyl-3-trimethylammonium-propane), DDAB (dimethyl-dioctadecylammonium bromide) and DODMA (1,2-dioleyloxy-3-dimethylaminopropane).

9. The liposome-based drug carrier for immune anticancer therapy according to claim 8, wherein the cationic lipid and the siRNA are loaded such that the ratio of the amino group of the cationic lipid and the phosphate group of the siRNA is 1:1 to 6:1.

10. A method for preparing a liposome-based drug carrier for immune anticancer therapy, comprising:
(a) a step of preparing a cationic liposome comprising siRNA, which is an immunosuppressive protein inhibitor, and a micelle;
(b) a step of forming a liposome particle comprising the immunosuppressive protein inhibitor siRNA, with a predetermined size, by mixing and sonicating the cationic liposome and the micelle, and then extruding the same;
(c) a step of preparing an aptamer-conjugated micelle by reacting an aptamer which is a cancer cell target-specific ligand with the micelle; and
(d) a step of mixing the aptamer-conjugated micelle, the liposome particle containing the immunosuppressive protein inhibitor siRNA, and an immunogenic chemo-anticancer agent.

11. The method for preparing a liposome-based drug carrier for immune anticancer therapy according to claim 10, wherein, in the step (a), the cationic liposome comprising the immunosuppressive protein inhibitor siRNA is prepared by hydrating a dry lipid membrane comprising a cationic lipid with a buffer comprising the immunosuppressive protein inhibitor siRNA.

12. The method for preparing a liposome-based drug carrier for immune anticancer therapy according to claim 11, wherein the cationic lipid and the siRNA is used such that the ratio of the amino group of the cationic lipid and the phosphate group of the siRNA is 1:1 to 6:1.

13. The method for preparing a liposome-based drug carrier for immune anticancer therapy according to claim 10, wherein the step (d) is performed at 50 to 70° C.

14. An anticancer agent composition comprising the liposome-based drug carrier for immune anticancer therapy according to claim 1.

15. The anticancer agent composition according to claim 14, wherein the anticancer agent composition is for treating any cancer selected from breast cancer, lung cancer, colorectal cancer, stomach cancer, liver cancer, gallbladder cancer, pancreatic cancer, cervical cancer, leukemia, lymphoma and prostate cancer.
